(19) United States
(12) Patent Application Publication
(10) Pub. No.: US 2008/0183095 A1
(43) Pub. Date: Jul. 31, 2008

(54) INFANT MONITOR

(76) Inventors: Colby R. Austin, Twin Falls, ID (US); Chris E. Curtis, Pocatello, ID (US); Jeff L. Otto, Moscow, ID (US); Jennifer L. Hasenoehrl, Lewiston, ID (US); Bart Semmler, Chugiak, AK (US); Lloyd C. Daugherty, Ridgecrest, CA (US); Laila Cornwall, Moscow, ID (US); Richard B. Wells, Moscow, ID (US)

Correspondence Address: KLARQUIST SPARKMAN, LLP, 121 SW SALMON STREET, SUITE 1600, PORTLAND, OR, 97204, US

(21) Appl. No.: 12/022,088

(22) Filed: Jan. 29, 2008

Related U.S. Application Data
(60) Provisional application No. 60/897,945, filed on Jan. 29, 2007.

(51) Int. Cl. A61B 5/08

(52) U.S. Cl. 600/534

(57) ABSTRACT

A breathing monitor and method for monitoring respiration, such as for detecting apnea events and/or preventing Sudden Infant Death Syndrome includes one or more variable inductance sensors that are configured to stretch and contract in response to breathing movements. Stretching and contraction are associated with an inductance change in the sensors, which are configured to alter a frequency of an oscillator circuit. The breathing monitor may also comprise a transmitter circuit coupled to a microcontroller or other processor that analyzes the frequency changes and sounds an alarm in the event that breathing ceases for a predetermined time period. The breathing monitor and associated sensor circuitry can be secured to a garment to be worn by an infant or other subject to be monitored.

### CROSS REFERENCE TO RELATED APPLICATION

The present application claims the benefit of the earlier filing date of U.S. Provisional Application No. 60/897,945, filed Jan. 29, 2007, which is incorporated herein by reference.

### FIELD

The present disclosure relates to breathing monitors.

### BACKGROUND

Several different breathing or respiration monitors have been developed to detect interrupted respiration. Such monitors have been used to prevent Sudden Infant Death Syndrome (SIDS), or for studying and treating sleep apnea in infants and adults. The currently available breathing monitors are typically expensive to manufacture, complex to operate and are not generally suitable for consumer use.

Some prior art breathing monitors use a microphone to detect the sounds of the breath. Other prior art breathing monitors detect changes in pressure in the airway. Still other prior art breathing monitors detect movement associated with breathing and can include pads that are placed under an infant's mattress. See, for example, U.S. Patent Application Publication 2004/0111039 A1. However such monitors can be affected by changes in pressure unrelated to breathing, such as changes in pressure caused by a ceiling fan, or the like.

U.S. Pat. No. 3,782,368 discloses a transducer construction and system for measuring respiration including an elastic belt and a piezoelectric element for obtaining accurate respiration data. Col. 1, line 7, 48, 56-57. U.S. Pat. No. 5,295,490 discloses an apnea monitor including a “belt means for substantially encircling a portion of the body of the patient and for expanding and contracting in response to respiration of the patient.” Col. 3, lines 24-26. In one embodiment of the '490 patent, “the belt means includes a substantially inextensible biased wire extending along at least a portion thereof . . . carried within a helical spring.” Col. 3, lines 29-31, 47-48. “Displacement of the wire cause by breathing is registered as an electrical signal. Col. 3, lines 66-68.

U.S. Pat. No. 4,494,553 discloses a vital signs monitor “which detects vital signs, such as the patient's breathing, by changing inductance.” Col. 1, line 55. “The patient unit also includes a mounting means, such as a belt or vest,” and a “transmitter of the patient unit transmits radio signals indicative of the patient's vital signs.” Col. 1, lines 56-60. The '553 patent discloses the use of “a plurality of inductive coils or loops 12 and 14,” where “the coils 12 and 14 move with respect to each other, causing a change in the mutual or relative inductance of these coils.” Col. 2, lines 30-31; col. 3, lines 1-3.

U.S. Pat. No. 4,433,693 is directed to a method and assembly for monitoring respiration and detecting apnea. The '693 patent discloses “the use of remote monitoring with a passive circuit means” which is “placed about the infant's chest by means of a band and the infant is thereafter placed within a radio frequency electromagnetic field.” Col. 1, line 62 to col. 2, line 2. “The expansion and contraction of chest 16 of baby 18 causes the band 14 which is positioned about the infant's chest to move the dielectric element 32 between the plates 34 of the capacitor 28 . . . to thereby vary the resonant frequency of the passive circuit means 12.” Col. 5, lines 14-21.

Some prior art monitors comprise articles of wearing apparel. For example, U.S. Pat. No. 5,454,376 discloses “a breathing monitor article of wearing apparel, adapted for child users.” Abstract. “An elastic belt extends about the chest and/or abdomen portion of the user” and “a strain gauge is secured to the elastic belt and detects breathing movement through the expansion and contraction of the chest wall.” Abstract. Also, U.S. Pat. No. 6,687,523 discloses “a garment for infants” with “a plurality of signal transmission paths integrated within.” Abstract.

Other monitors continuously measure “variations in the patient's chest cross sectional area . . . by measuring the inductance of an extensible electrical conductor closely looped around the body, by connecting the loop as the inductance in a variable frequency LC oscillator followed by a frequency-to-voltage converter and voltage display.” U.S. Pat. No. 4,815,473, Abstract. Still other breathing monitors use ultrasound and are not approved for home use.

Monitors that comprise an elastic or inelastic strap that encircles the chest and/or abdomen can be uncomfortable, especially for infants. Such straps may easily be pushed out of place which can affect monitoring accuracy and reliability. Furthermore, these straps typically include sensors that directly contact the infant's skin. While these devices may be safe, many parents are uncomfortable with electronics that directly contact their children.

In view of the above, a need remains for accurate, inexpensive breathing monitors that can be conveniently used by consumers and medical professionals.

### SUMMARY

Systems and methods for monitoring respiration, such as for detecting apnea events are disclosed. The disclosed systems and methods of the present disclosure do not require loops or straps that completely encircle the torso of the wearer, and can be more comfortable and accurate than conventional systems. In some examples, breathing monitors comprise one or more variable inductance sensors that can expand or contract due to expansion and contraction of a wearer's chest. Sensor expansion and contraction is associated with changes in inductance of the one or more variable inductance sensors. The one or more sensors are coupled to one or more sensor oscillators such that variation in the inductance of the sensors can alter the oscillation frequencies of the one or more sensor oscillators. The sensor oscillators and one or more frequency comparators are configured to detect associated frequency shifts. The frequency comparators are coupled to a transmitter that is configured to communicate frequency shifts to a base unit comprising a microcontroller or other similar device. The base unit is configured to monitor breathing based on the received frequency shifts. Visible and/or audible alarms are coupled to the base system, and can be activated as needed. For example, the base unit can sound an alarm in the event that breathing stops for a predetermined period of time.

Variable inductance sensors can be integrated into garments such as infant sleepwear and can be connected to garments with button snaps or VELCRO® fasteners so as to be easily removable. Other electronic components can be removably attached so that sensors and electronic components can be removed to permit washing, or to attach to a different garment. In some examples, such breathing monitors can be configured to consume little power, so that extended operation is possible with batteries.

One embodiment of a breathing monitor comprises a garment, at least one impedance sensor configured to be secured to the garment and situated on the garment so as to be responsive to breathing, and a breathing detector removably attached to the garment and configured to provide a breathing status indication based on an impedance of the at least one sensor. The impedance sensor is a variable inductance sensor in some examples, and one, two, three, four, or more sensors may be present. In some embodiments with four sensors, two pairs of sensors are connected in series, and each pair is connected in parallel.

The breathing monitor may comprise at least one snap fastener configured to removably attach the breathing detector to the garment, wherein the snaps are configured to electrically connect the breathing detector and the at least one impedance sensor. Additionally, the breathing monitor may be housed in a flexible plastic protective enclosure.

The garment can be a suitable garment for infants, such as an undergarment or pajama. Additionally, the garment can comprise a stabilizer fabric and a fabric overlayer, and a variable inductance sensor may be located between the stabilizer fabric and the overlayer. The garment may have a midline extending vertically along the length of the garment as situated upright, and the first and second variable inductance sensors can be positioned on the garment to the right of the midline, and the third and fourth variable inductance sensors can be symmetrically positioned on the garment to the left of the midline. The variable inductance sensors may each be positioned substantially horizontally with reference to the vertical midline of the garment.

The breathing monitor may comprise a sensor oscillator configured to be coupled to the at least one impedance sensor and to provide a sensor oscillator signal at a sensor frequency associated with an impedance of the at least one impedance sensor, a reference oscillator configured to produce a reference oscillator signal at a reference frequency, and a frequency comparator configured to produce a frequency comparator signal associated with a difference between the sensor frequency and the reference frequency, and a processor configured to produce a breathing status indication based on the difference. In some embodiments, the sensor oscillator comprises a modified Colpitts oscillator that includes a transistor having an emitter and a fixed inductor and a resistor placed in series between the emitter and a ground connection.

A variable inductance sensor can comprise an elastic core and an inductor comprising first and second ends configured for coupling to a breathing monitor, first and second anchor portions secured to the elastic core, and a coil about the elastic core situated between the first and second anchor portions. The sensor can further comprise stitched regions configured to secure the first and second anchor portions to the elastic core.

A garment suitable for monitoring respiration in an infant can comprise at least four variable inductance sensors, an inner chest panel, an outer chest panel, and a breathing detector positioned between the inner and outer chest panel. A French seam can be configured to secure the at least four variable inductance sensors.

A method for monitoring respiration in a subject can comprise securing one or more variable inductance sensors and a breathing detector to a garment, electrically connecting the one or more variable inductance sensors and the breathing detector, detecting a change in frequency associated with a change in inductance of the one or more variable inductance sensors, and analyzing the detected frequency and providing an indication of subject respiration based on the detected frequency change. The method can also include displaying a signal indicative of subject respiration and/or sounding an audible alarm in response to the indication.

Another method for monitoring respiration in a subject can comprise positioning first, second, third, and fourth inductive coils substantially perpendicular to a subject vertical midline extending across a chest of the subject such that the first and second inductive coils are positioned near an upper chest region of the subject and are symmetrically situated about the vertical midline, and the third and fourth inductive coils are positioned near a diaphragm region of the subject and are symmetrically situated about the midline, detecting an inductance change in at least one of the inductive coils in response to breathing movements of the subject, and transmitting a respiration indication in response to a detected inductance change.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 shows a simplified plan/block diagram view of a system for monitoring breathing according to the present disclosure.

FIG. 2 is a simplified plan view of one embodiment of a variable inductance sensor for monitoring breathing according to the present disclosure.

FIG. 3 is a simplified plan view of a garment that includes a plurality of variable inductance sensors.

FIG. 4 is a simplified plan view of a garment that includes a plurality of variable inductance sensors.

FIG. 5A is a block diagram of a representative breathing detector.

FIG. 5B is a block diagram of a representative base unit.

FIG. 6 is a schematic electrical circuit diagram of a modified Colpitts oscillator according to the present disclosure.

FIG. 7 is a schematic electrical circuit diagram of one arrangement of multiple variable inductance sensors.

FIG. 8 is a schematic electrical circuit diagram of one embodiment of an oscillator circuit combined with transmitting circuitry.

FIG. 9 is a plan view of a garment with an integrated breathing monitor system according to the present disclosure.

FIG. 10 is a plan view of the garment of FIG. 9, with certain panel portions removed.

FIG. 11 is a plan view of a garment with an integrated breathing monitor system according to the present disclosure.

FIG. 12 is a block diagram of a method of monitoring respiration.

### DETAILED DESCRIPTION

As used in this application and in the claims, the singular forms “a,” “an,” and “the” include the plural forms unless the context clearly dictates otherwise. Additionally, the term “includes” means “comprises.”

The described systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods which function in the manner described by such theories of operation.

As used herein, a signal is a constant or time varying electrical voltage or current. Electrical components are conveniently referred to as being “connected” or “coupled,” but unless otherwise specified or apparent, such coupling does not exclude the presence of intermediate elements.

### System

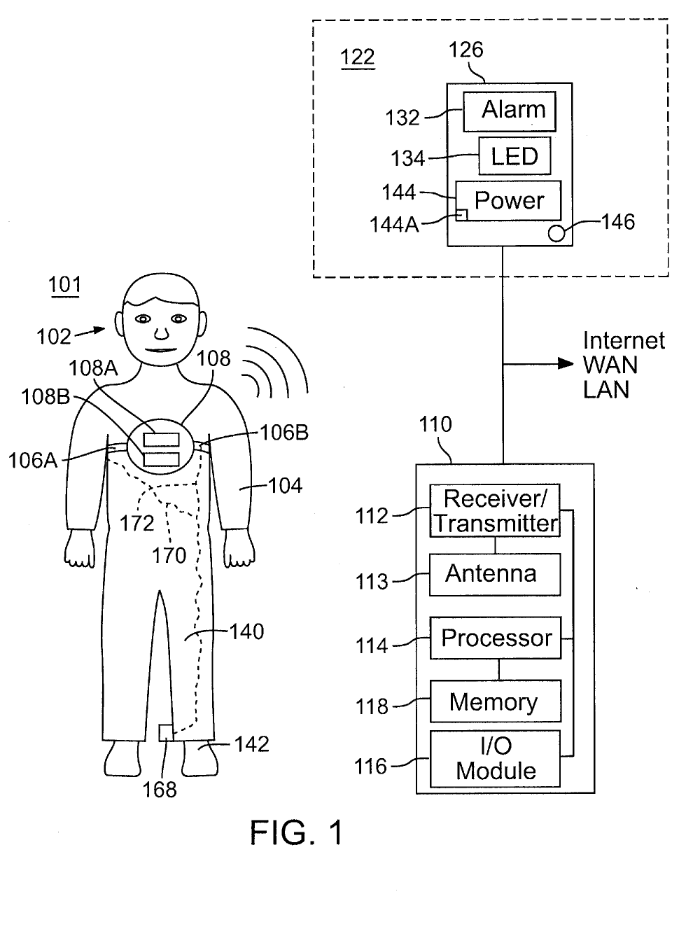

FIG. 1 shows one embodiment of a system for monitoring breathing according to the present disclosure. An infant 102 is fitted with a garment 104 that includes variable inductance sensors 106A, 106B. The variable inductance sensors 106A, 106B are electrically coupled to a breathing detector 108 that is removably attached to the garment 104, such as by VELCRO hook and loop fasteners or some other suitable fasteners. The breathing detector 108 includes at least one frequency comparator 108A and a transmitter 108B configured to wirelessly communicate breathing status as digital data to a base unit 110.

Sensors, such as the variable inductance sensors 106A, 106B are configured to change inductance in response to expansion and contraction of the wearer's chest in the normal course of breathing, and are generally referred to as variable inductors. An inductor is usually constructed of conducting material, such as copper wire, that generally is coiled, looped, or wrapped around a core of air, a ferromagnetic material, or other materials. Core materials with greater permeabilities provide increased inductance.

The base unit 110 can include a receiver/transmitter 112, that is coupled to an antenna 113, a processor 114 for analyzing received data, and a memory 118 for data storage or storage of computer-executable instructions for analysis or other processing or communication. The memory 118 can be ROM, RAM, a hard disk, or other storage components or combinations thereof. An input/output module 116 is configured to communicate via a local area network (LAN) or a wide area network (WAN) such as the Internet, or with wired or wireless telephone networks. Alternatively, the receiver/transmitter 112 can be configured to communicate via a network, or a separate wireless network module can be provided.

The base unit 110 can be coupled to a remote receiver 126 that can be located in a remote location 122 such as in a separate room in a house. For example the garment 104, variable inductance sensors 106A, 106B, the breathing detector 108, and the base unit 110 can be located within an infant's bedroom 101, while the remote receiver 126 is located in a separate room, such as a living area or a parent's bedroom. The remote receiver 126 may comprise one or more audible alarms 132, and/or one or more warning lights or other visual alarms 134. Warning alarms and/or lights also may be used to indicate low battery life, breathing irregularities, or loss of connectivity between one or both of the variable inductance sensors 106A, 106B and the breathing detector 108. Electronics within the base unit 110 may control the alarm(s) and/or light(s) on the remote receiver 126, based on input signals received from the breathing detector 108.

In other embodiments, the base unit 110 may be unnecessary, as base unit functions may be included within the breathing detector 108. Additionally, in some embodiments, the breathing detector 108, the base unit 110, and/or the remote receiver 126 may be coupled to a network, and breathing status indicated on in-home monitors or display units, or communicated via the Internet or other networks. In some of these embodiments, a breathing monitor or breathing detector may communicate with wireless devices, such as a laptop or a wireless or cellular phone. In some embodiments, alarms may be provided in e-mails, text messages, or vibrations received in devices provided to parents, family members, caregivers, and/or doctors or other health personnel.

In another example shown in FIG. 1, a breathing detector 168 can be attached to the garment 104 near an ankle portion 142, and the variable inductance sensors 106A, 106B coupled to the breathing detector 168 with respective conductors 170, 172 that can be woven into or otherwise secured to the garment 104.

As shown in FIG. 1, in some embodiments, the breathing detector 108 may be directly connected to the variable inductance sensors 106A, 106B. In some other embodiments, a breathing detector (such as the breathing detector 168) can be distant from the sensors 106A, 106B. For example, the breathing detector 168 can be located near the ankle 142 of the garment 104. The variable inductance sensors 106A, 106B can be electrically coupled to the breathing detector 108 with additional lengths of the wire used to form the variable inductance sensors 106A, 106B. For example, the variable inductance sensors 106A, 106B may comprise sufficient wire to travel down a leg portion 140 of the garment 104 so as to connect to the breathing detector 168 located near the ankle portion 142 of the garment 104. These additional wires (for example, the conductors 170, 172) may be concealed, such as sewn into a French seam, so as to be hidden and inaccessible to the infant or other wearer.

Although the FIG. 1 examples include two sensors 106A, 106B, one, two, three, four, or more sensors may be used. Other embodiments comprise different numbers of variable inductance sensors, such as four variable inductance sensors positioned in various configurations, some of which will be described below.

Some embodiments of a remote receiver 126 can comprise circuitry similar to that of a base unit 110. The remote receiver 126 can comprise a power supply 144, power switch 144A, speaker 132, and volume control 146. The remote receiver 126 may be designed to operate and appear to be similar to a standard baby monitor and can include functionality similar to a standard baby monitor. A microphone (not shown in FIG. 1) can be integrated into the breathing detector 108 and/or the base unit 110, thus allowing the breathing monitor to additionally operate as an intercom.

Some embodiments of remote receivers such as the receiver 126 provide for distinctly different audible alarms depending on the situation. For example, a remote receiver 126 may provide an alarm when the batteries are low, and this alarm may sound substantially different from a second alarm indicating cessation of breathing. The remote receiver 126 may optionally provide other alarms such as an alarm indicating that one or more variable inductance sensors is disconnected. Each of these alarms may be distinct, and corresponding distinct visible alarms can be provided in addition to or instead of the audible alarms.

### EXAMPLE SENSORS

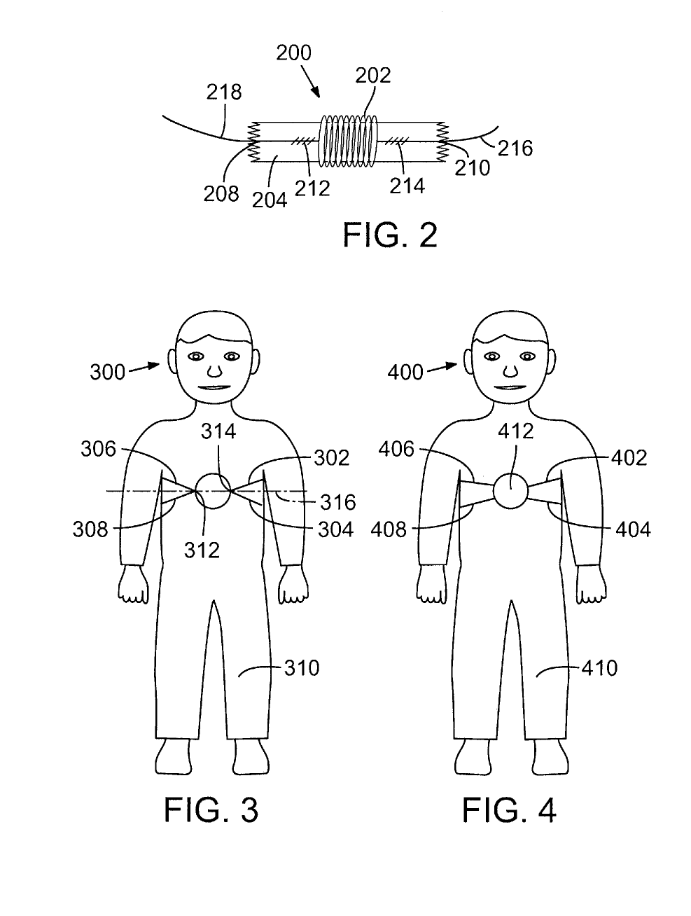

In one example shown in FIG. 2, a variable inductor sensor 200 comprises a coil 202 of enamel coated wire or other conductor wrapped around a core material 204. Alternatively, in some embodiments, sensors may be shielded or may comprise wire coils completely or partially enclosed in ferrite or other high permeability material. Some representative sensors comprise a fabric or other protective material surrounding a variable inductor so as to prevent fabric from the garment from being trapped by the coils. Inductor conductors (typically wire) can be any conductor sufficiently flexible to be looped tightly enough to give a measurable change in inductance when a coil is stretched or contracted by breathing movements. For example, enameled wire can be used such as enamel-coated wire manufactured and distributed by Infantron Singapore Pte. Ltd. Some embodiments comprise very thin wires (i.e. wires with a very small diameter), or wires that can be submerged in water for washing or otherwise cleaned. In one embodiment, 32 gauge enameled copper wire is used, but even smaller diameter wire and wires of different alloys can be used to provide increased flexibility while maintaining strength. In some embodiments, a low friction material surrounds the coiled wire and is configured to decrease the likelihood of the wire coil being caught or hung up on a garment. Some representative variable inductance sensors are hand- or machine-washable so that sensors need not be removed from a garment before washing.

Referring further to FIG. 2, end portions 208, 210 of the conductive coil 202 are secured to the elastic core 204, but remaining portions of the coil can move freely. Such a configuration is more easily stretched than some other variable inductors, such as those comprising wire stitched in a zigzag pattern to an elastic base material. A wire coil about an elastic core can be more stable than a stitched zigzag inductor.

In some examples, the elastic core 204 can be formed of an elastic strip, such as a nylon cord, rayon cord, twisted cord, elastic cord, silk cord, elastic trim, elastic binding, elastic string, elastic webbing, elastic straps, elastic yarn, or any stretchable or contractible material. A particular configuration can be selected as needed. For example, the elastic core 204 can be an elastic strip approximately three inches long. The conductive coil 202 can be formed of enameled copper wire or other conductor and the first end portion 208 and the second end portion 210 can be configured to be electrically connected to breathing monitor circuitry. The coil 202 also comprises anchor portions 212, 214 that are woven, threaded into, or stitched in place along the core 204, such as to secure the coil 202 to the core 204. In one embodiment, the first anchor portion 212 can be anchored to the core 204 about one inch from the end of the core 204. In typical examples, sensor coils or sensor cores are between about 0.1 and 10 inches long and cores are between about 0.1 and 5 inches wide.

Once the first anchor portion 212 is secured, the conductor can then be coiled or wound around and along the length of the elastic strip 204 towards the second anchor portion 214 to form the coil 202. Coiling the conductor as tightly and closely as possible may result in improved characteristics for the variable inductance sensor 200. In one embodiment, the electrical conductor is coiled until the coil 202 is about one inch long. Single or multiple layers of the conductor can be used to form the coil 202. Then, the second anchor portion 214 can be threaded into, stitched in place, or otherwise secured to the elastic strip 204, so that the coil 202 stretches and contracts as the elastic strip core stretches and contracts. The number of turns and length of the coil can be varied to achieve desired measures of inductance for a particular variable inductance sensor. One example sensor can comprise from about forty to about fifty-five turns, and its inductance may range from about one to about four microhenrys when stretched and contracted. Sensor coils can be circular, elliptical, oval, rectangular, or other shapes as may be convenient.

As shown in FIG. 3, variable inductance sensors 302, 304, 306, 308 are positioned on each side of an infant, i.e. the sensors 302, 304 are positioned near the left chest and armpit region, while the sensors 306, 308 are positioned near the right chest and armpit region. The right two sensors 306, 308 can be positioned to form a “V” shape, with the “V” opening laterally away from the midpoint of the chest. Similarly, the left two sensors 302, 304 can be positioned to form a “V” shape, with the “V” opening laterally away from a midpoint of the chest. Vertices 312, 314 of the sets of sensors can be at substantially the same height along the length of the infant 300 or garment 310, i.e., the vertices 312, 314 can be situated substantially on an axis 316 that is horizontal with the garment 310. This positioning can allow the variable inductance sensors 302, 304, 306, 308 to provide a conveniently large inductance change in response to wearer respiration movements.

FIG. 4 illustrates an alternative breathing monitor that includes a breathing detector 412 and variable inductance sensors 402, 404, 406, 408 that are secured to a garment 410. In the configuration shown in FIG. 4, each of the sensors 402, 404, 406, 408 extends substantially horizontally from a breathing detector 412 located on the garment 410. If four sensors are used, the sensors 402, 406 may be located nearer to a wearer's head 400 that the sensors 404, 408. Also as seen in FIG. 4, the sensors 406 and 408 may be electrically coupled to each other in series, and the sensors 402 and 404 may also be electrically coupled to each other in series. Each pair of the sensors 402, 404, 406, 408 can be electrically coupled to the breathing detector 412. Sensor coils are typically situated and configured to permit expansion and contraction during respiration. For example, a longitudinal axis of a sensor coil (an axis about which a coil is formed) is aligned with a direction of movement during respiration.

The representative embodiments described above comprise variable inductance sensors. It should be understood that other types of sensors may also be provided. For example, some embodiments may comprise one or more variable resistance or variable capacitance sensors. In some embodiments, different types of sensors may be combined within the same system.

### Representative Circuitry

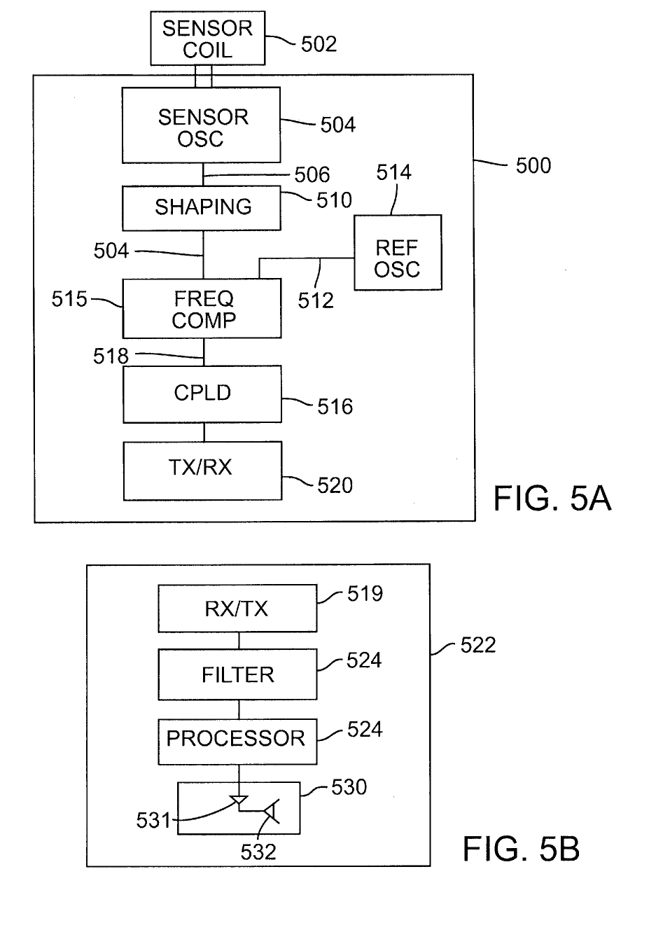

FIG. 5A is a block diagram of a representative breathing detector 500. A variable inductance sensor 502 (or a plurality of such sensors) is situated at or on a subject and configured to exhibit a change in inductance as a result of extension and contraction of an inductive coil due to motion of a subject's chest and/or abdomen during breathing. The variable inductance sensor 502 is coupled to a sensor oscillator circuit 504 that produces a sensor oscillator signal at an output 506 at a frequency associated with an inductance of the variable inductance sensor 502. A change in inductance of the variable inductance sensor 502 alters the output frequency of the sensor oscillator circuit 504. In some embodiments, the sensor oscillator signal is coupled to an input 508 of a waveform shaping comparator 510. The comparator 510 is coupled to the output 506 of the sensor oscillator circuit 504, and is configured to shape the sensor oscillator signal, typically to produce a square wave waveform. In other embodiments, the waveform shaping comparator 510 is not used.

In the illustrated embodiment, a frequency comparator 515 is coupled to a sensor oscillator output 506 (or a waveform shaping comparator output 518) and an output 512 from a reference oscillator 514 to produce an electrical signal associated with a difference between a reference oscillator signal and a shaped or unshaped sensor oscillator signal, typically based on a frequency difference. In other examples, oscillator amplitude, quality factor (Q), spectral width, or other oscillator characteristic can be used. In other examples, a reference oscillator is not used and sensor oscillators associated with different sensors are used to establish one or more frequency differences. The frequency comparator 515 is coupled to a complex programmable logic device 516 at an output 518. The complex programmable logic device 516 is configured to analyze the incoming signal (typically a time-varying frequency difference) and deliver breathing monitor data based on the incoming signal to a transmitter 520. The transmitter 520 may be a digital or analog radio frequency transmitter, an infrared transmitter, or other transmitter.

Referring to FIG. 5B, the transmitter 520 can, in one representative example, wirelessly send information to a receiver 519 (or transceiver) located in a base unit 522, typically located remotely from the transmitter 520. The breathing monitor data received by the base unit 522 can optionally be filtered by a digital filter 524 and coupled to a microcontroller 528. The microcontroller 528 is configured to process the filtered data according to predetermined programmed criteria, determine whether breathing conditions appear abnormal, and activate an alarm 530, if desired. Typically, the microcontroller 528 is electrically coupled to an amplifier 531 and speaker 532 to sound an alarm.

In alternative embodiments, the elements present in FIGS. 5A-5B need not all be included. For example, the transceiver 522 may be unnecessary, or may be located within the breathing detector 500. In some embodiments, the waveform shaping comparator 510 may be unnecessary, or equivalent functionality may be provided by the complex programmable logic device 516 or some other device. Additionally, warning lights, such as light emitting diodes (LEDs) or other visual display(s) may be present on the breathing detector 500 and/or the transceiver 522. The breathing detector 500 may comprise an alarm, amplifier, and/or speaker similar to the alarm 530, amplifier 531, and speaker 532 as shown within the transceiver 522. Digital filtering may be included in the CPLD 516, and memory used to store computer or processor-executable instructions and/or data is not shown for convenience.

The sensor oscillator 504 can be configured to generate a signal at a base frequency designed to minimize or reduce interference with other devices commonly found in residential or commercial settings. The base frequency is typically within a range of between about 2 MHz and about 5 MHz. Alternatively, a lower base frequency can be used, such as a frequency of about 2 MHz or less, or a frequency of about 1 MHz or less. In other examples, a frequency between about 10 kHz and 10 GHz can be used as may be convenient. The sensor oscillator base frequency may be tunable by input from a user, so that if a certain frequency is experiencing interference, a different base frequency can be used. The base frequency is varied according to the changing inductance of the variable inductance sensors 502, and the sensor oscillator and sensor inductance are configured to provide a convenient base frequency.

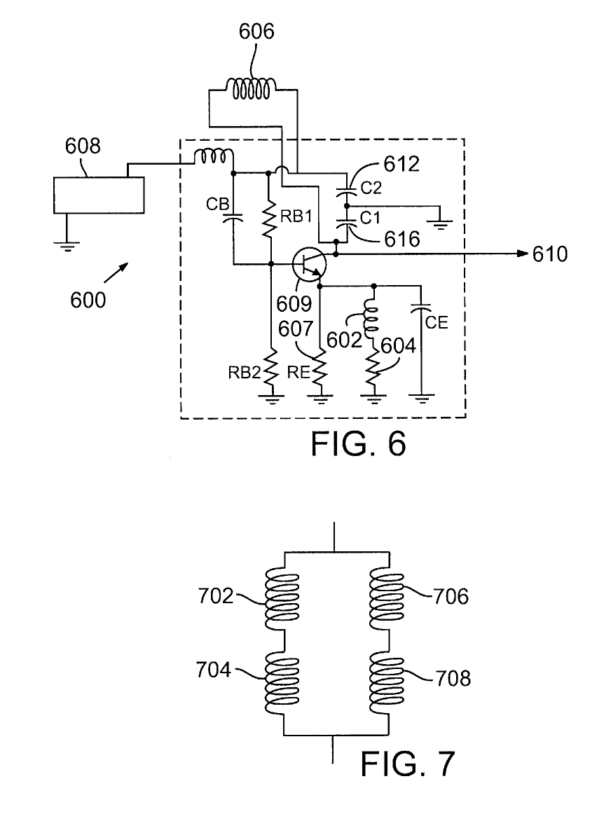

While there are many suitable oscillator circuits, in one example a modified Colpitts oscillator is used. FIG. 6 is a schematic electrical circuit diagram of a modified Colpitts oscillator 600 that includes a fixed inductor 602 and a resistor 604 that are situated to, in conjunction with resistor 607, establish DC and AC bias for a bipolar transistor 609. Additionally, the modified Colpitts oscillator of FIG. 6 includes a variable inductance sensor 606, such as those illustrated above. Oscillator frequency is dependent on inductance of the variable inductance sensor 606 and capacitance values of capacitors 612, 614.

The fixed inductor 602, in combination with resistor 604 tends to provide relatively stable oscillator output without consuming substantial power. The resistor 604 is placed in series with the inductor 602 and limits current from a power source 608. The inductor 602 and the resistor 604 can be associated with a surprisingly large voltage swing at an output 610 of the oscillator circuit 600, thus increasing breathing monitor sensitivity. The output 610 of the oscillator 600 can be coupled to an input of a waveform shaping comparator or a frequency comparator.

One or more variable inductance sensors such as the sensor 606 may be used. In embodiments that include two or more variable inductance sensors, a sensor oscillator such as the oscillator 600 can be configured in a variety of ways. For example, each of the variable inductance sensors may be linked in series. In other embodiments, each of the variable inductance sensors may be linked in parallel. In still other embodiments, the variable inductance sensors may be configured such that some are in parallel, while others are in series. For example, FIG. 7 shows a configuration that includes four variable inductance sensors 702, 704, 706, 708. A first pair of variable inductance sensors 702, 704 is coupled in series, a second pair of variable inductance sensors 706, 708 is also coupled in series, and the two pairs are connected in parallel. In some embodiments, more than one sensor oscillator can be provided and each of the variable inductance sensors 702, 704, 706, 708 may be connected to an independent sensor oscillator, and each sensor oscillator can be coupled to comparators or processors as may be convenient. In some embodiments, one or more switches may be provided, so that the sensor oscillator is selectively coupled to different sensors or different pairs or other grouping of sensors. Breathing events can be detected based upon frequency shifts or other oscillator characteristics that are typically associated with inductance changes of less than about 0.1%, 0.2%, 0.4%, or 1.0%.

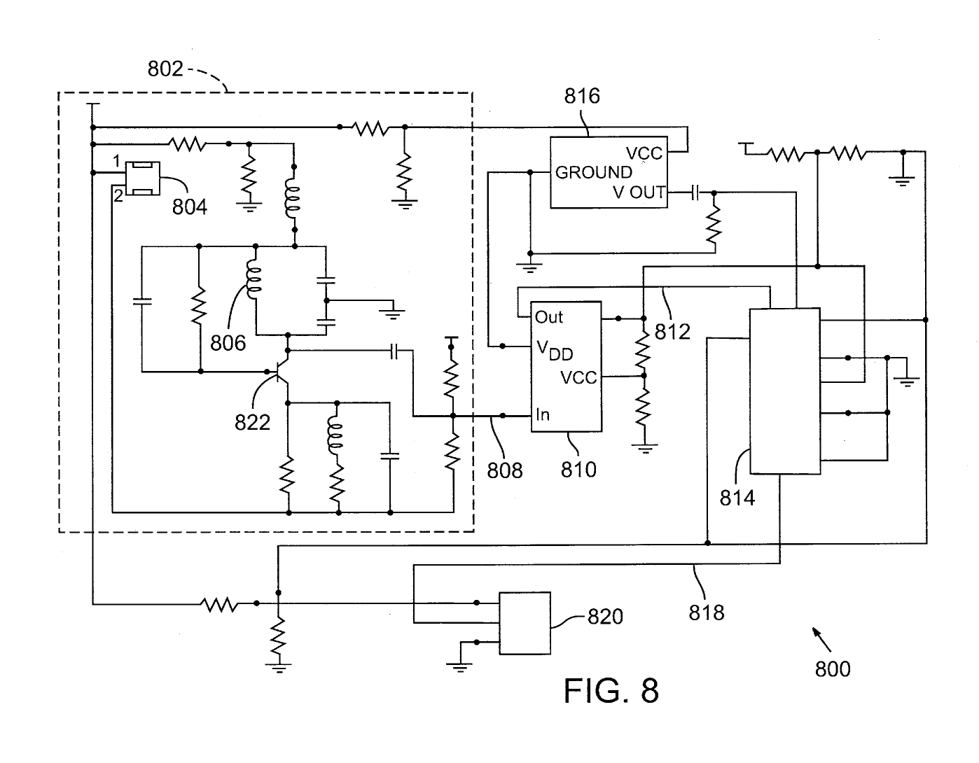

FIG. 8 is a simplified schematic diagram of one embodiment of a breathing monitor 800 that includes a sensor oscillator circuit 802 integrated with other circuit elements, for use in a system for monitoring breathing. Circuit elements with multiple input/output connections have been simplified such that not all connections are shown for clarity. The breathing monitor 800 includes a battery or other power source 804 and one or more variable inductance sensors 806. An oscillator output 808 of the sensor oscillator circuit 802 can be coupled to a frequency comparator 810 that is configured to process a signal based on a difference frequency associated a difference between a reference signal from a reference oscillator 816 and an output signal from the sensor oscillator 808.

In one embodiment, a frequency comparator output 812 is coupled to a complex programmable logic device (CPLD) 814 or other processing circuitry. The complex programmable logic device 814 may also be coupled to the reference crystal oscillator 816. In alternative embodiments, the frequency comparator 810 and the CPLD 814 can be replaced with a single device, such as a field programmable gate array, or other similar device. Whether the CPLD 814 is used in conjunction with the comparator 810, or a field programmable gate array or other similar device is used, one suitable method for analyzing the sensor oscillator output comprises determining a difference between the sensor oscillator output frequency and a reference frequency, such as provided by a reference crystal oscillator or a clock device.

An output 818 of the CPLD 814 can be connected to a transmitter 820 for wireless communication with a transceiver located in a base unit and/or a remote receiver. In one embodiment, the transmitter 820 is a digital transmitter, such as an Xbee™ digital transmitter or a Bluetooth® based transmitter. A signal from the transmitter 820 can be received by a corresponding transceiver on a base unit and/or remote receiver, such as an Xbee™ transceiver or Bluetooth® transceiver. This signal can then be digitally filtered or otherwise processed and coupled to a microcontroller or other processor.

One method for analyzing a signal received from such a transmitter comprises using a calibration table stored in a memory coupled to a microcontroller to determine when the incoming frequency has been in a steady state for a predetermined time period, typically ten, twenty, thirty or more seconds. The predetermined time period can be based on standard definitions of apnea events or other breathing standards depending on the particular application for the breathing monitor. In this embodiment, if the microcontroller identifies a cessation of breathing for at least the predetermined time period, a local alarm is generated to alert or awaken the wearer. The microcontroller also can activate a transmitter on the breathing monitor or on a base unit in order to send an alarm signal to a remote receiver.

In alternative embodiments, a breathing detector located on an infant's garment can include both a microcontroller and a transmitter such that a separate base unit is unnecessary. In this embodiment, the breathing detector is configured to communicate directly with a remote receiver or other wireless device. Upon receiving a notification that a breathing disturbance has occurred, the remote receiver can sound an alarm. In some embodiments, the transmitter may transmit information at predetermined intervals, such as once per minute to indicate that the monitoring system is still working, and that breathing is normal. In some embodiments, the transmitter may be activated only to indicate that a breathing disturbance has been detected. In other embodiments, the transmitter can transmit information each time a breath is detected or can substantially continuously communicate breath-related information or other information such as information regarding the remaining breathing detector battery power.

Many variations on the disclosed circuitry are available. The described embodiments are not meant to limit the use of alternative electronic components and circuit designs. For example, the breathing detector may comprise an oscillator, an amplifier output stage, a battery or other power source, and an antenna. In alternative embodiments, the breathing detector need not have an antenna. Also, various elements may be exchanged for one another. For example, a bipolar transistor is shown in FIG. 8. However, alternative embodiments may comprise different elements and/or different types of transistors, such as field-effect transistors, op-amps, or other active or passive circuit elements.

The breathing detector, base unit, and/or remote receiver may comprise a power switch, or an on/off switch or button, or other similarly functioning components. Power may be provided by one or more batteries, such as a rechargeable lithium ion battery, and/or power may be provided from another source such as an AC adapter. Users may be able to set various monitoring options. For example, the user may be able to alter the time period defining an apnea event. Users may also be able to adjust the volume of any audible alarms provided by the breathing detector, base unit, and/or remote receiver. Users may be provided with a way of adjusting the sensitivity of the variable inductance sensors and/or the base frequency of the oscillator circuit. In some embodiments, visible alarms or indicators may be provided on the breathing detector, base unit, and/or remote receiver. Such visible alarms may indicate apnea events, low battery power, breath status, other system or breathing conditions, and/or a disconnection within the system. In other embodiments, visible light indicators may illuminate upon each detected breath.

Disclosed embodiments of an oscillator circuit and a breathing detector can operate at low voltage and low current. In one example, a voltage of less than 1.09 V and a current of about 0.3 mA can be used.

If batteries are used, the base unit and/or transmitting circuitry may comprise an alarm to provide an alert associated with remaining battery life. The device may optionally contain a battery life indicator which can give information as to how much battery life remains. Additionally, a battery power alert may sound differently than a breathing-related alarm. A breathing monitor may also comprise a warning alarm and/or light which would sound or turn on in the event that one or more variable inductance sensors is disconnected.

The disclosed circuits for use with a breathing monitor can optionally include other components including those configured to reduce or eliminate external interference and environmental noise.

The variable inductance sensors, oscillator circuit, comparator, reference oscillator, CPLD, and/or the transmitter can be configured to be secured to a particular garment, or configured to attach to any garment. In alternative embodiments, at least some of these components may be located on or remotely from a subject to be monitored.

### EXAMPLE GARMENTS

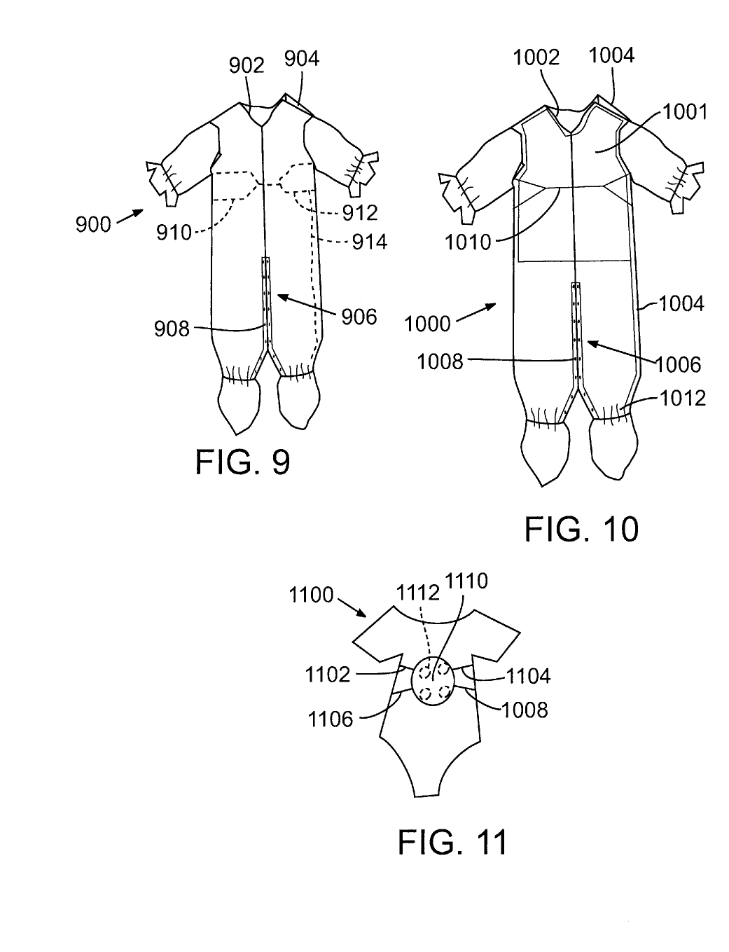

Some embodiments of a system for monitoring respiration comprise a garment to be worn by a human, for example, a garment fitted for an infant. One example of such a garment is garment 900 illustrated in FIG. 9. The garment 900 may comprise several design features to aid in functionality and comfort. A neckline 902 or top portion of the garment 900 may be closed by buttons 904. Alternatively, the neckline 902 of the garment 900 may be closed by one or more snaps, hook and loop fasteners (e.g. Velcro® fasteners), one or more zippers, or any other suitable closures. Inside leg seams 906 similarly may be closed with snaps 908 or other design element for easy donning and access. Sensors 910, 912 can be secured to or within the garment 900, and a conductor 914 provided for connection to a breathing detector located at an ankle portion of the garment.

Some embodiments of suitable garments comprise a full body garment, such as an infant's one-piece sleeping garment that comprises openings for the infant's head and hands but otherwise covers the infant. Alternative embodiments comprise an infant undergarment. Garment-based mounting has several advantages, especially for infants. For example, one or more variable inductance sensors can be associated with a garment, and then concealed, such as by panels of fabric or French seams, so that the infant cannot access the sensors.

Additionally, in some embodiments of a garment, connecting wires from the variable inductance sensors to the oscillator circuit and/or transmitter are concealed and are stitched in or on the garment, so that the wires do not interfere with infant movement, and to prevent the infant from gaining access to the wires. Such a garment may comprise an opening near at least one portion of the garment near the wearer's ankle or foot, allowing the connecting wires to exit the garment to connect to a transmitter. The transmitter may be secured to a garment exterior and encased in a housing to prevent access to the transmitting circuitry. Suitable housings may have exteriors in the form of a foot rattle, stuffed animal, or toy that can be connected to an ankle or foot portion of the garment.

Suitable materials for making such garments include stretch knits, cotton interlocks, jerseys, lightweight double knits, velour, and combinations thereof. Other embodiments of a garment can comprise any fabric or material, or combinations of fabrics or materials, suitable for making clothing or garments. In some embodiments, the garment is styled to be relatively tight fitting, so that sensors attached to the garment can expand or contract with wearer breathing.

In some embodiments, a garment may serve as a housing for variable inductance sensors, an oscillator circuit, a transmitter or transceiver, and/or a device such as a complex programmable logic device, a field programmable gate array, and/or a microcontroller. For example, the sensors and any circuitry or electronic components can be located on or in the infant's garment. In some embodiments, variable inductance sensors can be located on and attached to the garment at or near a chest portion of the garment. In some embodiments, circuitry can be attached to the garment, or enclosed within a portion of the garment. In other embodiments, transmitting circuitry may be secured to a garment exterior just outside the garment.

In some embodiments, one or more variable inductance sensors are attached to or enclosed within a garment, and the sensors are secured to a strip or other portion of a stabilizer fabric such as a fusible, non-fusible, or adhesive-backed stabilizer fabric. Suitable stabilizer fabrics can comprise a stiffer fabric than is conventionally used in infant garments. In some embodiments, a stabilizer fabric may comprise areas or portions of stiffness, such that the stabilizer fabric exhibits little or no stretch in response to expansion and contraction of the chest. Use of a stabilizer fabric can help secure sensors in place at a location associated with a preferred range of motion during breathing to provide sensor coil stretching/contraction during use. Such use can also result in a greater stretch exhibited in the variable inductance sensors, because the fabric itself will not expand with expansion of the chest. Stabilizer fabric can be secured to an inside layer of the garment to cover at least a portion of the chest region. Sensors secured to stabilizer fabric and suitably positioned tend to be responsive to breathing with reduced response to other movements and remain in a preferred location.

In some embodiments, one or more variable inductance sensors can be associated with the garment, whether or not the garment comprises a layer of stabilizer fabric. Flexible elastic webbing placed, for example, under arm portions of the garment can be used to connect the rear and front sections of the garment. Some embodiments comprise a flexible elastic webbing, or other suitable material with a low stiffness and/or low elastic modulus such that it stretches easily. In some embodiments, the garment itself or portions thereof can be less flexible than the elastic webbing used to support one or more variable inductance sensors.

FIG. 10 illustrates a representative garment 1000 with an exterior chest panel removed to show underlying structures. The garment 1000 may comprise the exterior chest panel (not shown) and an interior chest panel 1001 that is configured to contact the wearer's chest and can be slightly smaller than the overlying exterior chest panel of the garment 1000. Hardware, such as a breathing detector, variable inductance sensors 1010, and an electrical connection 1004, can be positioned between the interior and exterior chest panels. After any hardware is placed as needed, seams can be sewn to enclose the hardware with, for example, French seams.

The electrical connection 1004 from the variable inductance sensors 1010 and/or a breathing detector can be collected at one side of the garment 1000 and sewn directly into the seam using a French seam to enclose the electrical connection 1004 such that any associated wires, conductors, or other components are inaccessible. In some embodiments, hardware for the breathing detector, such as connecting wires and/or circuit elements, is positioned between the inner and outer layers of fabric, and sewn inside a seam along an outside leg portion of the garment, exiting the garment near an ankle portion 1012. A detachable monitor unit (not shown) may be connected to the garment 1000 near the ankle portion 1012, and electrically connected using wires which exit the garment. Electrical connection between any hardware located on or within the garment, such as the variable inductance sensors, and a detachable hardware unit can be provided using wires in combination with snap or other connections such as, for example, a nine volt battery contact snap connector.

FIG. 11 shows an alternative garment 1100 for use with an infant wearer. The garment 1100 is configured as an infant undergarment to be worn under normal infant sleepwear or clothing so as to restrict infant contact with breathing monitor hardware. Variable inductance sensors 1102, 1104, 1106, 1108 can be configured on the garment 1100 so as to provide stretching and contraction of the variable inductance sensors 1102, 1104, 1106, 1108 in response to breathing. In the illustrated embodiment, the sensors 1102, 1104 are placed in a substantially horizontal position near the upper chest region of the garment 1100 with the garment 1100 in an upright position. The sensors 1106, 1108 are positioned substantially horizontally and located at a side wall region so to be at or near the infant's diaphragm.

The garment 1100 may be provided with breathing detector 1110 that includes electronic circuit elements such as sensor and/or reference oscillators, a transmitter/transceiver, and/or a logic or microcontroller device. Such electronic circuit elements may be contained within a flexible plastic pouch that is water resistant and/or resistant to tearing and puncture. The breathing detector 1110 may be removably connected to the garment 1100 using any suitable method, such as by using snap connectors 1112. In some embodiments, the snap connectors 1112 can provide electrical connections between electronic circuit elements contained within breathing detector 1110 and the variable inductance sensors 1102, 1104, 1106, 1108.

Four variable inductance sensors 1102, 1104, 1106, 1108 are shown in FIG. 11. In alternative embodiments, more or fewer variable inductance sensors may be provided. Additionally, the variable inductance sensors 1102, 1104, 1106, 1108 are positioned substantially horizontally. In other embodiments, the variable inductance sensors 1102, 1104, 1106, 1108 may be positioned at one or more angles tilted from horizontal. Alternatively, the variable inductance sensors 1102, 1104, 1106, 1108 may be positioned substantially vertically, or configured along with other sensors arranged substantially vertically. In some embodiments, some variable inductance sensors can be positioned substantially vertically and between two or more variable inductance sensors positioned substantially horizontally on the garment.

A transmitter can be sheltered inside a small anklet so as to be concealed from view and to limit access to hardware by the wearer. One suitable method for concealing transmitting circuitry comprises placing the circuitry within a rattle or a stuffed animal which is then attached to the garment or to the foot or ankle of the infant or other wearer. A detachable unit such as described can be secured to the ankle with any suitable fastener such as, for example, with Velcro® straps. If desired, a second similarly weighted unit may be placed on an opposite ankle or foot, in order to provide a balanced experience for the wearer.

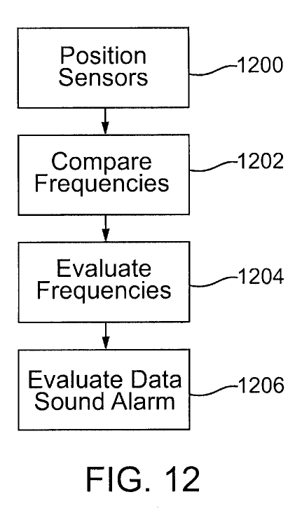

With reference to FIG. 12, a representative breathing detection method includes a step 1200 of situating one or more variable impedance sensors at suitable locations on a subject. The variable impedance sensors can be configured to provide a variable resistance, inductance, or capacitance in response to subject chest, diaphragm or other movement associated with breathing. The sensors can be conveniently situated by securing the sensors to a garment worn by the subject. In a step 1202, one or more resonant frequencies or oscillation frequencies associated with the one more variable impedance sensors are compared with one or more reference frequencies that are typically provided by a crystal-based reference oscillator to provide a difference frequency that is associated with an impedance change responsive to subject breathing. In a step 1204, the difference frequency is processed to provide a breath status indication that is associated with an extent of breathing (i.e., how deeply the subject is breathing) at a particular time. In some examples, the breathing extent is not obtained, but only an indication of the presence or absence of a breath. Typically, breath status indications are recorded and stored in a memory. In a step 1206, breathing extent (or presence or absence) as a function of time is evaluated to determine if an alarm is to be sounded or otherwise indicated. Typically, breathing extent or presence is evaluated over a selected time period of between about ten (10) seconds and sixty (60) seconds. In some examples, an alarm is associated with breathing cessation or with a breathing irregularity such as change in breathing depth, frequency, or other breathing changes.

While certain embodiments of the disclosed subject matter have been described for use with infants or young children, the same technology can be easily adapted for other uses. For example, breathing monitors of the present disclosure can be used detecting apnea events in any patient. Breathing monitors can also be used to monitor respiration of athletes or elderly patients, other human subjects, or animal subjects.

In view of the many possible embodiments to which the principles of the disclosed technology may be applied, it should be recognized that the illustrated embodiments are only examples and should not be taken as limiting the scope of the technology. Rather, the scope of the technology is defined by the following claims. We therefore claim as our invention all that comes within the scope and spirit of these claims.

We claim:

1. A breathing monitor comprising:
a garment;
at least one impedance sensor configured to be secured to the garment and situated on the garment so as to be responsive to breathing; and
a breathing detector removably attached to the garment and configured to provide a breathing status indication based on an impedance of the at least one sensor.

2. The breathing monitor of claim 1, further comprising at least one snap fastener configured to removably attach the breathing detector to the garment, wherein the snaps are configured to electrically connect the breathing detector and the at least one impedance sensor.

3. The breathing monitor of claim 1, wherein the breathing detector comprises a sensor oscillator configured to be coupled to the at least one impedance sensor and to provide a sensor oscillator signal at a sensor frequency associated with an impedance of the at least one impedance sensor, a reference oscillator configured to produce a reference oscillator signal at a reference frequency, and a frequency comparator configured to produce a frequency comparator signal associated with a difference between the sensor frequency and the reference frequency, and a processor configured to produce a breathing status indication based on the difference.

4. The breathing monitor of claim 3, wherein the sensor oscillator comprises a modified Colpitts oscillator that includes a transistor having an emitter and a fixed inductor and a resistor placed in series between the emitter and a ground connection.

5. The breathing monitor of claim 3, where the at least one sensor is a variable inductance sensor.

6. The breathing monitor of claim 2, wherein the breathing detector is housed in a flexible plastic protective enclosure.

7. The breathing monitor of claim 1, wherein the garment is selected from the group consisting of an infant undergarment and an infant pajama.

8. The breathing monitor of claim 3, wherein the garment comprises a stabilizer fabric and a fabric overlayer, and the variable inductance sensor is located between the stabilizer fabric and the overlayer.

9. The breathing monitor of claim 5, wherein the at least one variable inductance sensor comprises first, second, third, and fourth variable inductance sensors.

10. The breathing monitor of claim 9, wherein the garment has a midline extending vertically along the length of the garment as situated upright and the first and second variable inductance sensors are positioned on the garment to the right of the midline, and the third and fourth variable inductance sensors are symmetrically positioned on the garment to the left of the midline.

11. The breathing monitor of claim 10, wherein the first and second variable inductance sensors are connected in series, and the third and fourth inductance sensors are connected in series.

12. The breathing monitor of claim 11, wherein the first and second variable inductance sensors are connected in parallel with the third and fourth variable inductance sensors.

13. The breathing monitor of claim 10, wherein the first, second, third, and fourth variable inductance sensors are each positioned substantially horizontally with reference to the vertical midline of the garment.

14. A variable inductance sensor, comprising:
an elastic core; and
an inductor comprising first and second ends configured for coupling to a breathing monitor, first and second anchor portions secured to the elastic core, and a coil about the elastic core situated between the first and second anchor portions.

15. The variable inductance sensor of claim 14, further comprising stitched regions configured to secure the first and second anchor portions to the elastic core.

16. A garment comprising:
at least four variable inductance sensors;
an inner chest panel;
an outer chest panel; and
a breathing detector positioned between the inner and outer chest panel.

17. The garment of claim 16, further comprising at least one French seam configured to secure the at least four variable inductance sensors.

18. A method for monitoring respiration in a subject, comprising:
securing one or more variable inductance sensors and a breathing detector to a garment;
electrically connecting the one or more variable inductance sensors and the breathing detector;
detecting a change in frequency associated with a change in inductance of the one or more variable inductance sensors; and
analyzing the detected frequency and providing an indication of subject respiration based on the detected frequency change.

19. The method of claim 18, further comprising displaying a signal indicative of subject respiration.

20. The method of claim 19, further comprising sounding an audible alarm in response to the indication.

21. A method for monitoring respiration in a subject, comprising:
positioning first, second, third, and fourth inductive coils substantially perpendicular to a subject vertical midline extending across a chest of the subject such that the first and second inductive coils are positioned near an upper chest region of the subject and are symmetrical situated about the vertical midline, and the third and fourth inductive coils are positioned near a diaphragm region of the subject and are symmetrically situated about the midline;
detecting an inductance change in at least one of the inductive coils in response to breathing movements of the subject; and
transmitting a respiration indication in response to a detected inductance change.
